(19) United States
(12) Patent Application Publication
(10) Pub. No.: US 2022/0064145 A1
(43) Pub. Date: Mar. 3, 2022

(54) SYNTHESIS OF POZIOTINIB DERIVATIVE

(71) Applicant: Hanmi Science Co., Ltd., Gyeonggi-do (KR)

(72) Inventors: Keuk Chan Bang, Gyeonggi-do (KR); Young Ho Moon, Gyeonggi-do (KR); Young Kil Chang, Gyeonggi-do (KR)

(73) Assignee: Hanmi Science Co., Ltd., Gyeonggi-do (KR)

(21) Appl. No.: 17/458,031

(22) Filed: Aug. 26, 2021

Related U.S. Application Data
(60) Provisional application No. 63/071,509, filed on Aug. 28, 2020.

(51) Int. Cl. C07D 401/14

(52) U.S. Cl. CPC C07D 401/14

(57) ABSTRACT

A method for the synthesis of a compound of Formula II is provided. Also disclosed is the salt form of the compound. The method includes the introduction of a piperidinyl moiety in a polar aprotic solvent system, followed by the removal of the protecting group and the acrylation step.

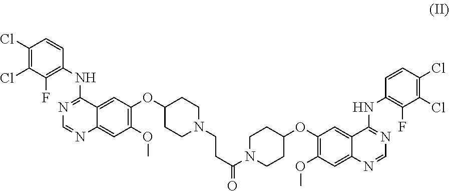 [SMILES: COc1cc2ncnc(Nc3ccc(Cl)c(Cl)c3F)c2cc1OC1CCN(CCC(=O)N2CCC(Oc3cc4c(Nc5ccc(Cl)c(Cl)c5F)ncnc4cc3OC)CC2)CC1]

### CROSS-REFERENCE TO RELATED APPLICATIONS

This application claims the benefit of U.S. Provisional Application 63/071,509 filed on Aug. 28, 2020. The entire contents of these applications are incorporated herein by reference in their entirety.

### TECHNICAL FIELD

Disclosed herein is a novel process for the preparation of a poziotinib derivative.

### BACKGROUND

Poziotinib, having a chemical name of 1-(4-(4-(3,4-dichloro-2-fluorophenylamino)-7-methoxyquinazolin-6-yloxy)piperidin-1-yl)prop-2-en-1-one, is known to exhibit anti-proliferative activities such as anti-cancer activities. The CAS Registry No. of the compound is 1092364-38-9.

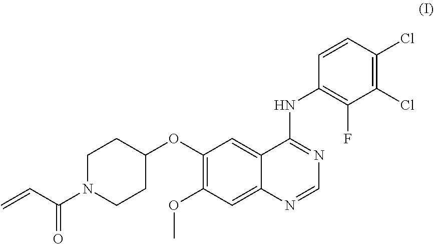 [SMILES: C=CC(=O)N1CCC(Oc2cc3c(Nc4ccc(Cl)c(Cl)c4F)ncnc3cc2OC)CC1]

While the synthesis of the above compound has been disclosed in international al patent application No. PCT/KR2014/000752, the preparation of its analogs or derivatives depends on the specific structure of the target compound and needs to be explored on a case-by-case basis.

### SUMMARY

This document discloses a novel synthesis of a quinazoline compound (Formula II) which bears two substituted quinazoline components. By employing suitable reaction conditions, the target compound can be generated as detailed herein.

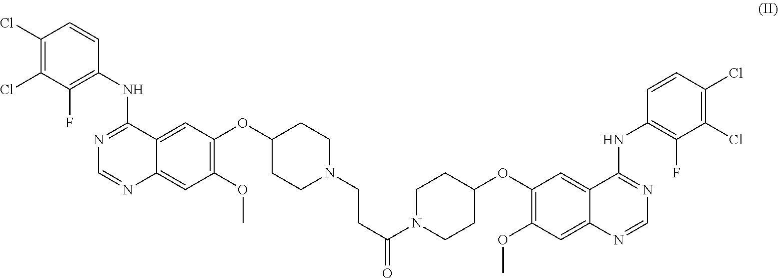 [SMILES: COc1cc2ncnc(Nc3ccc(Cl)c(Cl)c3F)c2cc1OC1CCN(CCC(=O)N2CCC(Oc3cc4c(Nc5ccc(Cl)c(Cl)c5F)ncnc4cc3OC)CC2)CC1]

An aspect of this patent document provides a method for preparing a compound of formula (II), which comprises the steps of:

(1) subjecting a compound of formula (VIII) to a reaction with a halogenating agent in the presence of an organic base, followed by a reaction with a compound of formula (X), to produce a compound of formula (VI);

(2) subjecting the compound of formula (VI) to a reaction with an ammonia solution in a polar protic solvent to produce a compound of formula (V);

(3) subjecting the compound of formula (V) to a reaction with a compound of formula (IX) in a polar aprotic solvent system in the presence of a base to produce a compound of formula (IV), wherein the polar aprotic solvent system comprises at least one selected from the group consisting of acetonitrile, acetone, dichloromethane, chloroform, carbon tetrachloride, 1,4-dioxane, ethyl acetate, tetrahydrofuran, and any combination thereof;

(4) subjecting the compound of formula (IV) to a reaction with an acid in an inert solvent to produce a compound of formula (III); and

(5) subjecting the compound of formula (III) to an acrylation reaction with

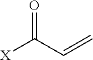

(wherein X is halogen) in the presence of a base to produce a compound of formula (II)

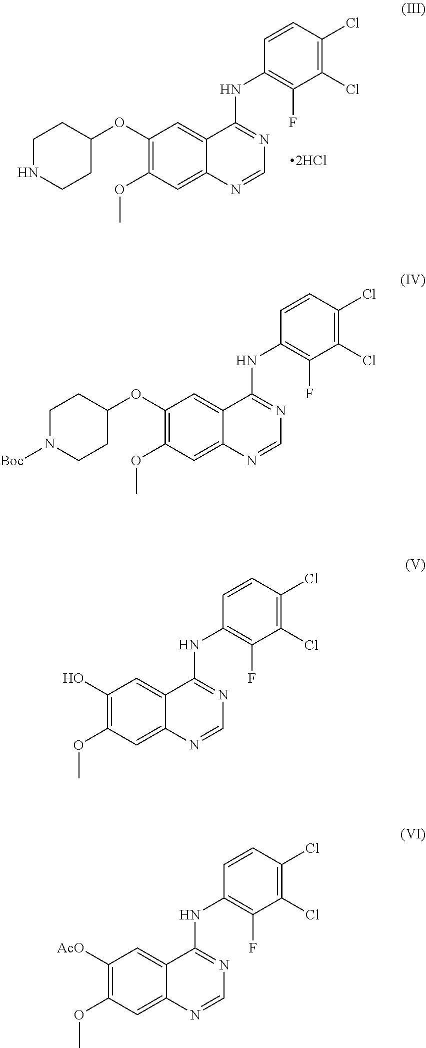 [SMILES: COc1cc2ncnc(Nc3ccc(Cl)c(Cl)c3F)c2cc1OC1CCNCC1 ; COc1cc2ncnc(Nc3ccc(Cl)c(Cl)c3F)c2cc1OC1CCN(C(=O)OC(C)(C)C)CC1 ; COc1cc2ncnc(Nc3ccc(Cl)c(Cl)c3F)c2cc1O ; COc1cc2ncnc(Nc3ccc(Cl)c(Cl)c3F)c2cc1OC(C)=O]

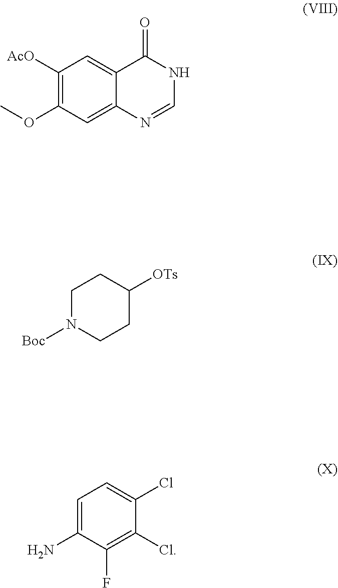 [SMILES: COc1cc2nc[nH]c(=O)c2cc1OC(C)=O ; Cc1ccc(S(=O)(=O)OC2CCN(C(=O)OC(C)(C)C)CC2)cc1 ; Nc1ccc(Cl)c(Cl)c1F]

In some embodiments, Step (1) is conducted in a solvent selected from the group consisting of toluene, benzene and a mixture thereof. In some embodiments, the organic base in Step (1) is selected from the group consisting of diisopropylamine, triethylamine, diisopropyl ethylamine, diethylamine, pyridine, 4-dimethylpyridine, morpholine and a mixture thereof. In some embodiments, the halogenating agent in Step (1) is selected from the group consisting of thionyl chloride, phosphorusoxy chloride and a mixture thereof.

In some embodiments, the polar protic solvent in Step (2) is selected from the group consisting of methanol, ethanol, propanol and a mixture thereof.

In some embodiments, the inert polar aprotic solvent in Step (3) further comprises a member selected from the group consisting of N,N-dimethylformamide, N,N-dimethylacetamide, N-methylpyrrolidin-2-one, dimethyl sulfoxide and a mixture thereof. In some embodiments, the base in Step (3) is an alkali metal carbonate salt selected from the group consisting of sodium hydrogen carbonate, potassium carbonate, cesium carbonate and a mixture thereof. In some embodiments, the base is employed in an amount of 1 to 5 mole equivalents based on 1 mole equivalent of the compound of formula (V). In some embodiments, Step (3) further comprises recrystallizing the compound of formula (IV) with acetone.

In some embodiments, the acid in Step (4) is employed in an amount of 3 to 10 mole equivalents based on 1 mole equivalent of the compound of formula (IV). In some embodiments, the acid of Step (4) is hydrochloric acid.

In some embodiments, Step (5) is conducted in an organic solvent selected from the group consisting of tetrahydrofuran, ethyl acetate, acetone, 1,4-dioxane, acetonitrile, dichloromethane, carbon tetrachloride, chloroform, N,N-dimethyl formamide and dimethylsulfoxide, or a mixture of said organic solvent and water. In some embodiments, the base in Step (5) is selected from the group consisting of sodium carbonate, calcium carbonate, potassium carbonate, sodium hydroxide, potassium hydroxide, cesium carbonate, diisopropylamine, triethylamine, diisopropylethylamine and diethylamine. In some embodiments, the base in Step (5) is employed in an amount of 3 to 5 mole equivalents based on 1 mole equivalent of the compound of Formula III.

In some embodiments, the method further includes acidifying the reaction mixture after the reaction and converting the reaction product to a salt form. In some embodiments, the salt form is obtained by reacting the compounds of Formula I and II with an acid selected from the group consisting of HCl, HBr, and MsOH.

### DESCRIPTION OF DRAWINGS

FIG. 1 shows ¹H-NMR of the compound of Formula II

FIG. 2 shows ¹³CNMR of the compound of Formula II

FIG. 3 shows ¹H-¹H COSY spectrum of the compound of Formula II

FIG. 4 shows ¹H-¹³C HSQC spectrum of the compound of Formula II

FIG. 5 shows ¹H-¹³C HMBC spectrum of the compound of Formula II

FIG. 6 shows ¹⁹F-NMR spectrum of the compound of Formula II

### DETAILED DESCRIPTION

This patent document discloses the novel synthesis of a compound of Formula II. The compound includes key structural components of poziotinib, which is known to exhibit anticancer activities. Through a beta elimination process, the compound of formula II can be converted to a compound of formula I and a quinazoline-containing moiety. The dual component feature of compound II may provide desirable pharmacodynamics and/or pharmacokinetic properties for therapeutic applications. The compound may also serve as a precursor to compound I and other compounds of therapeutic potential.

As used herein, the articles “a” and “an” refer to “one or more” or “at least one,” unless otherwise indicated. That is, reference to any element or component of an embodiment by the indefinite article “a” or “an” does not exclude the possibility that more than one element or component is present.

As used herein, the term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items. Expressions such as “at least one of,” when preceding a list of elements, modify the entire list of elements and do not modify the individual elements of the list. When referring to a dosing protocol, the term “day”, “per day” and the like, refer to a time within one calendar day which begins at midnight and ends at the following midnight.

The present method may be the carried out as shown in the schemes below. Steps (1) and (2) of the present method can be carried out in accordance with Reaction Scheme 1:

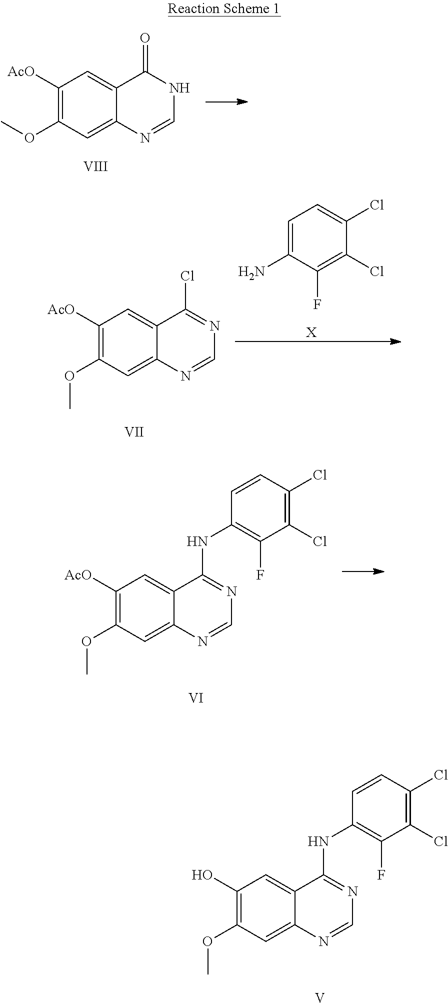

In Step (1), the compound of formula (VIII) as a starting material is subjected to a reaction with a halogenating agent in a solvent such as toluene or benzene in the presence of an organic base, followed by a reaction with the compound of formula (X), to produce 4-(3,4-dichloro-2-fluorophenylamino)-7-methoxyquinazolin-6-yl acetate of formula (VI). The compound of formula (VIII) can be prepared by the method disclosed in Korean Patent No. 1013319, the entire disclosure of which is incorporated herein by reference.

The organic base used in Step (1) of the present method may be selected from the group consisting of diisopropylamine, triethylamine, diisopropyl ethylamine, diethylamine, pyridine, 4-dimethylpyridine, morpholine and a mixture thereof; and the halogenating agent may be selected from the group consisting of thionyl chloride, phosphorusoxy chloride and a mixture thereof.

The reaction may be conducted at a temperature of 50° C. to 150° C., preferably 60° C. to 90° C., more preferably about 75° C. As a result of the reaction with the halogenating agent, the compound of formula (VII) may be prepared as contained in the organic solvent, which cannot readily be separated. Subsequently, the compound of formula (VII) contained in the organic solvent is subjected to a reaction with the compound of formula (X) to produce 4-(3,4-dichloro-2-fluorophenylamino)-7-methoxyquinazolin-6-yl acetate of formula (VI).

In Step (2), the compound of formula (VI) prepared in Step (1) is subjected to a reaction with an ammonia solution or ammonia gas in a polar protic solvent (e.g., methanol, ethanol and propanol) at a temperature of 0° C. to 40° C., preferably 10° C. to 30° C., more preferably about 25° C., to produce 4-(3,4-dichloro-2-fluorophenylamino)-7-methoxyquinazolin-6-ol of formula (V).

In Step (3), as illustrated in Reaction Scheme 2, the compound of formula (V) is subjected to a reaction with tert-butyl 4-(tosyloxy)piperidin-1-carboxylate of formula (IX) in an inert polar protic solvent in the presence of a base to produce tert-butyl 4-(4-(3,4-dichloro-2-fluorophenylamino)-7-methoxyquinazolin-6-yloxy)piperidin-1-carboxylate of formula (IV).

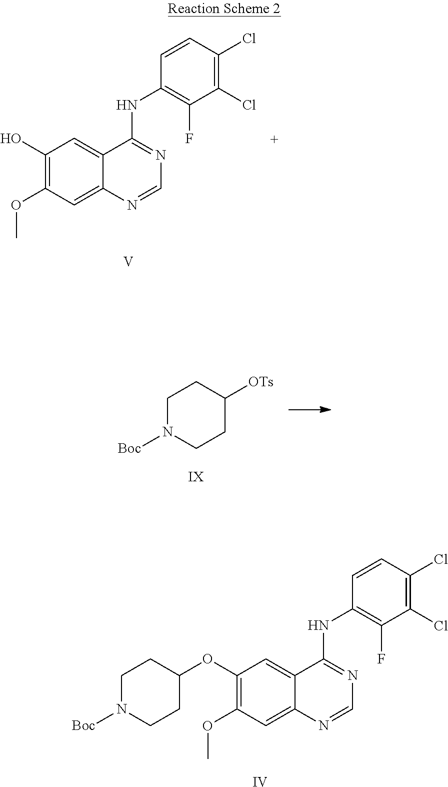

The inert polar aprotic solvent used in Step (3) of the present method may contain one or more of acetonitrile, acetone, dichloromethane, chloroform, carbon tetrachloride, 1,4-dioxane, ethyl acetate, tetrahydrofuran, and any combination thereof. In some embodiments, the inert polar aprotic solvent contains at least one of acetonitrile, acetone, dichloromethane, chloroform, carbon tetrachloride, 1,4-dioxane, ethyl acetate, and tetrahydrofuran. In some embodiments, the inert polar aprotic solvent may contain additionally one or more of N,N-dimethylformamide, N,N-dimethylacetamide, N-methylpyrrolidin-2-one, dimethyl sulfoxide and a mixture thereof.

The base may be an alkali metal carbonate salt selected from the group consisting of sodium hydrogen carbonate, potassium carbonate, cesium carbonate and a mixture thereof. The base is used in an amount of 1 to 5 mole equivalents based on 1 mole equivalent of the compound of formula (V). The reaction may be conducted at a temperature of 60° C. to 100° C., preferably 70° C. to 90° C., more preferably about 80° C.

In some embodiments, the compound of formula (IV) can be prepared in purified by simple recrystallization in acetone.

In Step (4), as depicted in Reaction Scheme 3, the compound of formula (IV) is subjected to a reaction with an acid in an inert solvent to produce the acid salt of N-(3,4-dichloro-2-fluorophenyl)-7-methoxy-6-(piperidin-4-yloxy)quinazolin-4-amine (formula III). Suitable acid includes, for example, hydrochloric acid, hydrobromic acid, trifluoracetic acid, and any combination thereof. In some embodiments, the acid is hydrochloric acid.

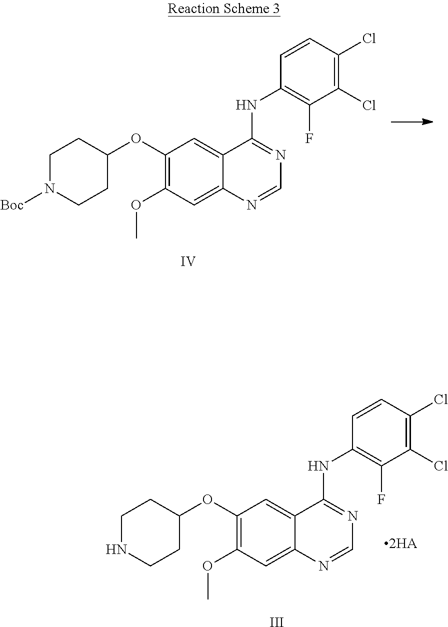

The inert solvent used in Step (4) of the present method may be selected from the group consisting of methanol, ethanol, propanol, ethyl acetate, methyl acetate, acetone and a mixture thereof. Hydrochloric acid may be used in an amount of 3 to 10 mole equivalents based on 1 mole equivalent of the compound of formula (IV). The reaction may be conducted under stirring for 1 to 24 hours at a temperature of 0° C. to 60° C., preferably 10° C. to 40° C., more preferably about 25° C.

In Step (5), as shown in Reaction Scheme 4, the compound of formula (III) is subjected to an acrylation reaction with

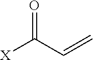

(wherein X is halogen), e.g., acryloyl chloride in the presence of a base to produce the compound of formula (II). In some embodiments, the acryloyl halide is added to a solution of the compound of formula (III).

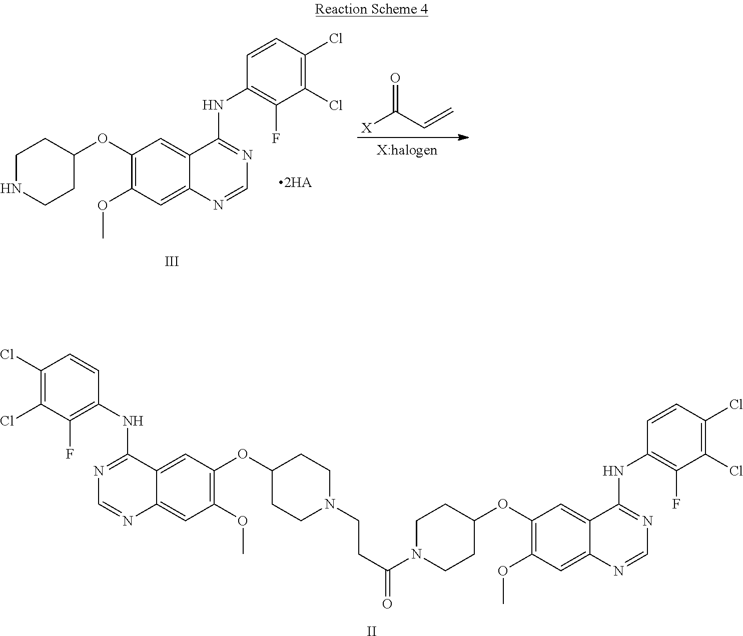

Step (5) of the present method can be conducted in an organic solvent such as tetrahydrofuran, ethyl acetate, acetone, 1,4-dioxane, acetonitrile, dichloromethane, carbon tetrachloride, chloroform, N,N-dimethyl formamide or dimethylsulfoxide, or in a mixture of said organic solvent and water. Preferred is a mixture of an organic solvent selected from the group consisting of tetrahydrofuran, ethyl acetate, acetone, 1,4-dioxane and acetonitrile, and water.

The base employed in Step (5) may be selected from the group consisting of an inorganic base such as sodium carbonate, calcium carbonate, potassium carbonate, sodium hydroxide, potassium hydroxide and cesium carbonate, and an organic base such as diisopropylamine, triethylamine, diisopropylethylamine and diethylamine. In this reaction, the base may be used in an amount of 3 to 5 mole equivalents based on 1 mole equivalent of the compound of formula (III). The reaction may be conducted under stirring for 20 minutes to 3 hours at a temperature of −30° C. to 20° C., preferably about 0° C.

The compound of formula II is obtained generally with the formation of a compound of Formula I. In some embodiments, both compounds coexist in the same mixture even after purification. The amount of the compound of formula II relative to the compound of Formula I may vary depending on the specific reaction condition such as the rate and amount of the acryloyl halide added to the solution of the compound of Formula III.

A related aspect of the patent document provides a composition of Formula II or a pharmaceutically acceptable salt thereof. The composition may contain one or more additional carriers. Acceptable additional carriers or diluents are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, glidants, dyes, fragrances, flavoring agents, and the like may be provided in the composition.

The following Examples are intended to further illustrate the present invention without limiting its scope.

### Example 1: Preparation of 4-(3,4-dichloro-2-fluorophenylamino)-7-methoxyquinazolin-6-yl Acetate (the Compound of Formula (VI))

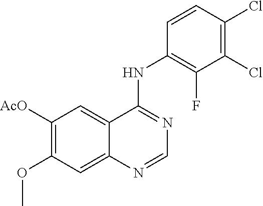 [SMILES: COc1cc2ncnc(Nc3ccc(Cl)c(Cl)c3F)c2cc1OC(C)=O]

7-methoxy-4-oxo-3,4-dihydroquinazolin-yl acetate (100 g) was added to toluene (850 ml) and N,N-diisopropylethylamine (82.5 ml). Phosphorusoxy chloride (100 ml) was added thereto over 20 minutes at 75° C., followed by stirring for 3 hours. Toluene (450 ml) and 3,4-dichloro-2-fluoroaniline (84.6 g) were added to the resulting mixture, followed by stirring for 2 hours. Upon completion of the reaction, the resulting mixture was cooled to 25° C. The solid thus obtained was filtered under a reduced pressure and washed with toluene (400 ml). Isopropanol (1,000 ml) was added to the solid, which was then stirred for 2 hours. The resulting solid was filtered and washed with isopropanol (400 ml). The solid was dried at 40° C. in an oven to produce the compound of formula (VI) (143 g, yield: 83%).

¹H-NMR (DMSO-d₆, 300 MHz, ppm) δ 8.92 (s, 1H), 8.76 (s, 1H), 7.69-7.57 (m, 3H), 4.01 (s, 3H), 2.38 (s, 3H).

### Example 2: Preparation of 4-(3,4-dichloro-2-fluorophenylamino)-7-methoxyquinazolin-6-ol (the Compound of Formula (V))

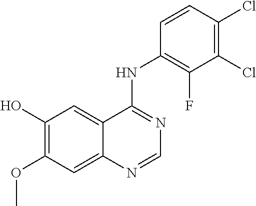 [SMILES: COc1cc2ncnc(Nc3ccc(Cl)c(Cl)c3F)c2cc1O]

4-(3,4-dichloro-2-fluorophenylamino)-7-methoxyquinazolin-6-yl acetate (100 g) was admixed with methanol (1,000 ml). The mixture was cooled to 10 to 15° C., added with an ammonia solution (460 g), and stirred for 3 hours at 25° C. The solid thus obtained was filtered and washed with a mixed solvent of methanol (200 ml) and water (200 ml). The resulting solid was dried at 40° C. in an oven to produce the compound of formula (V) (74 g, yield: 83%).

¹H-NMR (DMSO-d₆, 300 MHz, ppm) δ 9.57 (br, 2H), 8.35 (s, 1H), 7.68 (s, 1H), 7.61-7.52 (m, 2H), 7.21 (s, 1H), 3.97 (s, 3H).

### Example 3: Preparation of tert-butyl-4-(4-(3,4-dichloro-2-fluorophenylamino)-7-methoxyquinazolin-6-yloxy)piperidin-1-carboxylate (the Compound of Formula (IV))

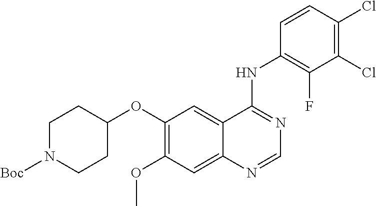 [SMILES: COc1cc2ncnc(Nc3ccc(Cl)c(Cl)c3F)c2cc1OC1CCN(C(=O)OC(C)(C)C)CC1]

4-(3,4-dichloro-2-fluorophenylamino)-7-methoxyquinazolin-6-ol (60 g) was admixed with N-dimethylformamide (360 ml) under stirring, followed by addition of tert-butyl 4-(tosyloxy)piperidin-1-carboxylate (120 g) and potassium carbonate (72 g) to the mixture. The reaction temperature was raised to 70° C., and the mixture was stirred for 14 hours. The temperature of the resulting solution was cooled to 25° C., and water (480 ml) was slowly added thereto. The solid thus obtained was filtered and dried. The solid was dissolved in a mixed solvent (600 ml) of dichloromethane and methanol. Active carbon (6 g) was then added thereto, followed by stirring for 30 minutes. The resulting mixture was filtered through a Celite pad, distilled under a reduced pressure, added with acetone (300 ml), and stirred for 2 hours. The resulting solid was filtered and washed with acetone (100 ml). The solid was dried at 40° C. in an oven to produce the compound of formula (IV) (75 g, yield: 83%).

¹H-NMR (DMSO-d₆, 300 MHz, ppm) δ 8.69 (s, 1H), 8.47 (t, 1H), 7.34-7.29 (m, 2H), 7.20 (s, 1H), 4.63-4.60 (m, 1H), 3.82 (s, 3H), 3.83-3.76 (m, 2H), 3.37-3.29 (m, 2H), 1.99-1.96 (m, 2H), 1.90-1.84 (m, 2H), 1.48 (s, 9H).

### Example 4: Preparation of N-(3,4-dichloro-2-fluorophenyl)-7-methoxy-6-(piperidin-4-yloxy)quinazolin-4-amine dihydrochloride (the Compound of Formula (III))

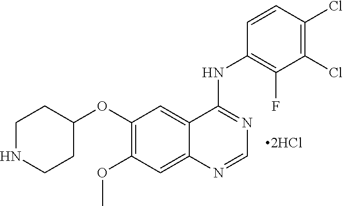 [SMILES: COc1cc2ncnc(Nc3ccc(Cl)c(Cl)c3F)c2cc1OC1CCNCC1]

Acetone (740 ml) was added to tert-butyl 4-(4-(3,4-dichloro-2-fluorophenylamino)-7-methoxyquinazolin-6-yloxy)piperidin-1-carboxylate (75 g), which was then stirred. The mixture was added with hydrochloric acid (145 ml) for 10 minutes and stirred for 5 hours. Upon completion of the reaction, the resulting mixture was filtered, and the solid thus obtained was washed with acetone (73 ml). The solid was dried at 30° C. in an oven to produce the compound of formula (III) (71 g, yield: 99%).

¹H-NMR (DMSO-d₆, 300 MHz, ppm) δ12.95 (bs, 1H), 9.42 (bs, 1H), 9.18 (bs, 1H), 9.01 (s, 1H), 8.86 (s, 1H), 7.69-7.56 (m, 2H), 7.45 (s, 1H), 5.11-5.08 (m, 1H), 4.03 (s, 3H), 3.29-3.20 (m, 4H), 2.33-2.30 (m, 2H), 1.96-1.93 (m, 2H).

### Example 5: Preparation of the Compound of Formula (II)

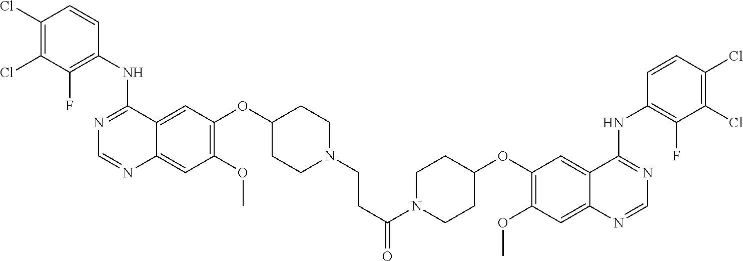 [SMILES: COc1cc2ncnc(Nc3ccc(Cl)c(Cl)c3F)c2cc1OC1CCN(CCC(=O)N2CCC(Oc3cc4c(Nc5ccc(Cl)c(Cl)c5F)ncnc4cc3OC)CC2)CC1]

N-(3,4-dichloro-2-fluorophenyl)-7-methoxy-6-(piperidin-4-yloxy)quinazolin-4-amine dihydrochloride (100 g) and sodium hydrogen carbonate (66 g) were added to a mixed solvent of tetrahydrofuran (630 ml) and water (1 L), and the temperature of the reaction mixture was cooled to 0° C. with iced water. Acryloyol chloride (24 ml) diluted with tetrahydrofuran (370 ml) was slowly added to the reaction mixture over 30 minutes, followed by stirring at 0° C. for 30 minutes. Upon completion of the reaction, aqueous acetone (2.0 L) was added to the resulting mixture, which was stirred for 12 hours and filtered to produce the compound of Formula II together with the compound of Formula I. The solid thus obtained was dissolved in a mixed solvent of dichloromethane (200 ml) and methanol (100 ml), added with ethyl acetate (1.2 L), and stirred for 12 hours. The resulting solid was filtered and washed with ethyl acetate (100 ml). The solid was dried at 40° C. in an oven to produce the compound of Formula II.

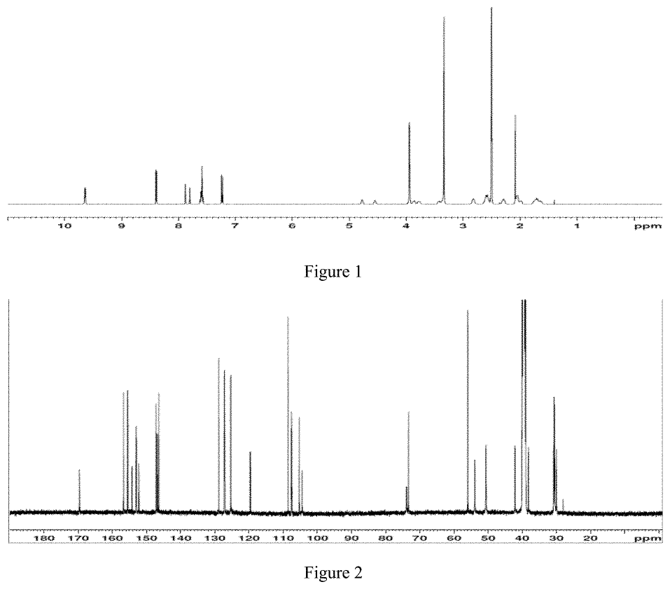

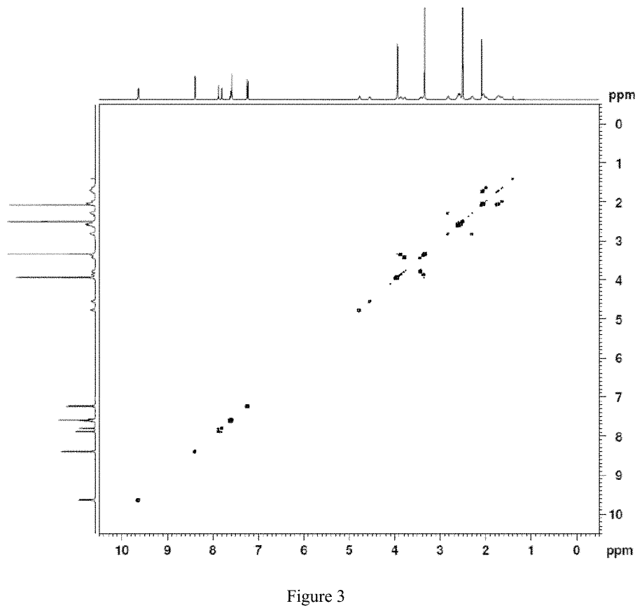

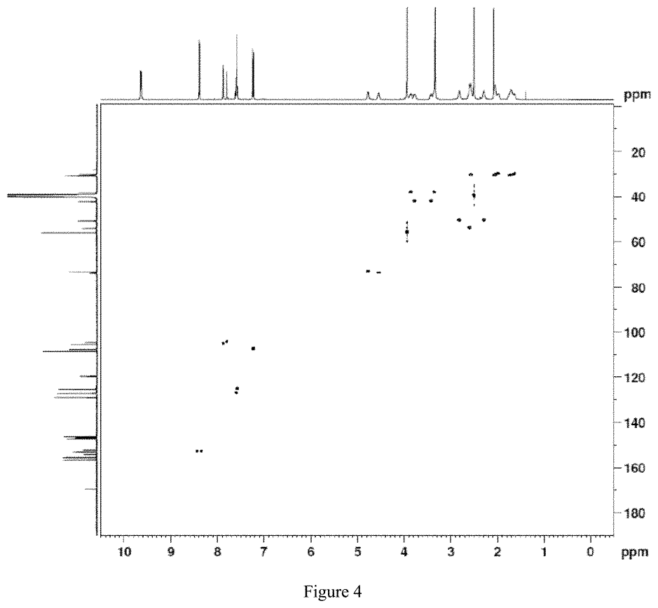

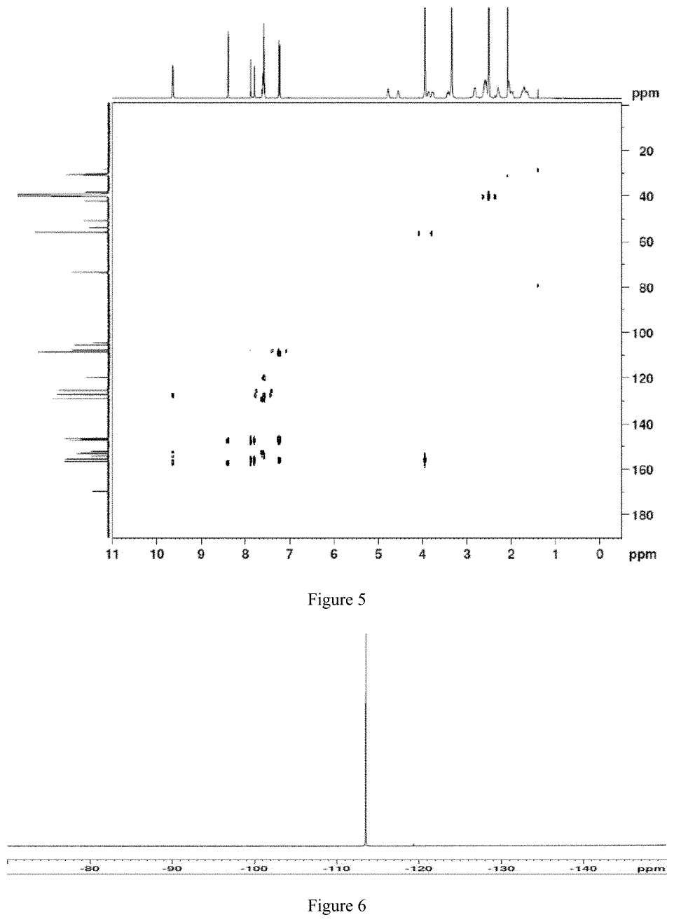

Table 1 shows total NMR chemical shift assignments for the compound of Formula II. Additional spectrum of the compound are shown in FIGS. 1-6.

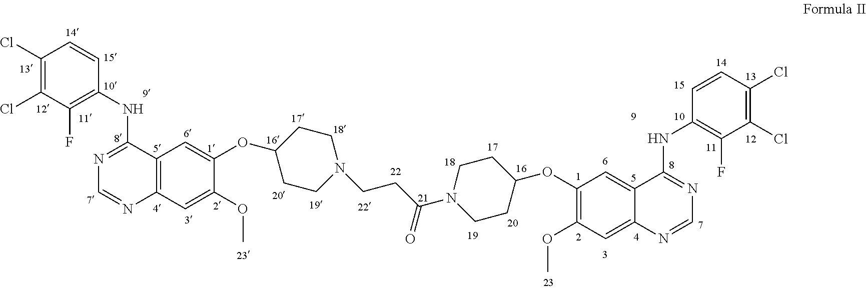 [SMILES: COc1cc2ncnc(Nc3ccc(Cl)c(Cl)c3F)c2cc1OC1CCN(CCC(=O)N2CCC(Oc3cc4c(Nc5ccc(Cl)c(Cl)c5F)ncnc4cc3OC)CC2)CC1]

TABLE 1
Total NMR chemical shift assignments
for the compound of Formula II.

| Position | | Chemical shift correlation result | |
|---|---|---|---|
| No. | Signal | δc | δ_{H} (Multiplicity) |
| 1, 1′ | C | 146.7, 147.0 | — |
| 2, 2′ | C | 155.4 (d) | — |
| 3, 3′ | CH | 104.5 | 7.80 (s) |
| | | 105.3 | 7.88 (s) |
| 4, 4′ | C | 146.4, 147.2 | — |
| 5, 5′ | C | 156.7 (d) | — |
| 6, 6′ | CH | 107.5 (d) | 7.22 (d) |
| 7, 7′ | CH | 152.9 (d) | 8.38 (d) |
| 8, 8′ | C | 108.6 | — |
| 9, 9′ | NH | — | 9.63 (d) |
| 10, 10′ | C | 128.8 | — |
| 11, 11′ | C | 152.2 (d), 154.2 (d) | — |
| 12, 12′ | C | 119.5 (d) | — |
| 13, 13′ | C | 127.2, 127.3 | — |
| 14, 14′, | CH | 125.3, 127.1 (d) | 7.56~7.62 (m) |
| 15, 15′ | | | |
| 16, 16′ | CH | 73.3 | 4.76 (m) |
| | | 73.9 | 4.55 (m) |
| 17, 17′, | CH₂ | 30.0 | 1.63~1.75 (m) |
| 20, 20 | | 30.8 | 1.99~2.06 (m) |
| 18, 18′, | CH₂ | 53.9 | 2.58~2.60 (m) |
| 19, 19′ | | 38.2 | 3.33~3.37 (m), 3.85~3.88 (m) |
| | | 42.2 | 3.42~3.44 (m), 3.76~3.79 (m) |
| 21 | C | 169.9 | — |
| 22, 22′ | CH₂ | 50.6 | 2.29 (m), 2.82 (m) |
| 23, 23′ | CH₃ | 55.9 (d) | 3.94 (d) |

In some embodiments, the synthesis further includes converting the product mixture of the compounds of Formula II and co-existing Formula I to a salt form. For instance, the product mixture can react with hydrochloric acid in an organic solvent (e.g., methanol, ethanol, propanol, isopropanol, butanol, ethyl acetate, acetone, tetrahydrofuran, acetonitrile, 1,4-dioxane and a mixture thereof) at a temperature of 0° C. to 60° C., preferably 10° C. to 40° C., more preferably at about 25° C. Other acidic agents that can be used for the preparation of the salt form include HBr, and MsOH.

The specific conditions such as reaction temperature, the amount of a reagent, and other relevant reaction factors in each of the above described reactions may vary. One skilled in art would be able to practive the synthesis methods of this patent document without undue experiments in view of the instant disclosure and the general knowledge in the field of organic chemistry. For instance, the preparation of certain intermediates of this patent document can be prepared in view of the examples of PCT/KR2014/000752, the entire disclosure of which is hereby incorporated by reference.

The activities of the compound of Formula II against targets such as EGFR can be evaluated according to various methods known in the art. Exemplary procedures are disclosed in U.S. Pat. No. 8,188,102, the disclosure of which is hereby incorporated by reference.

While the forgoing text may reference or exemplify specific embodiments of a reaction step or a method of preparing an intermediate, it is not intended to limit the scope of the method to such particular reference or examples. Various modifications may be made by those skilled in the art, in view of practical and economic considerations, such as the amount of the individual intermediates or reagent in the reaction and the length of time of conducting the reaction.

It will be appreciated by persons skilled in the art that invention described herein are not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific agent, or a step of the method, and may result from a different combination of described constituents, or that other un-described alternate embodiments may be available for a combination or method, is not to be considered a disclaimer of those alternate embodiments. It will be appreciated that many of those un-described embodiments are within the literal scope of the following claims, and others are equivalent.

1. A method for preparing a compound of formula (II), which comprises the steps of:
(1) subjecting a compound of formula (VIII) to a reaction with a halogenating agent in the presence of an organic base, followed by a reaction with a compound of formula (X), to produce a compound of formula (VI);
(2) subjecting the compound of formula (VI) to a reaction with an ammonia solution in a polar protic solvent to produce a compound of formula (V);
(3) subjecting the compound of formula (V) to a reaction with a compound of formula (IX) in a polar aprotic solvent system in the presence of a base to produce a compound of formula (IV), wherein the polar aprotic solvent system comprises at least one selected from the group consisting of acetonitrile, acetone, dichloromethane, chloroform, carbon tetrachloride, 1,4-dioxane, ethyl acetate, tetrahydrofuran, and any combination thereof;
(4) subjecting the compound of formula (IV) to a reaction with an acid (HA) in an inert solvent to produce a compound of formula (III); and
(5) subjecting the compound of formula (III) to an acrylation reaction with 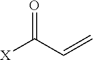
(wherein X is halogen) in the presence of a base to produce a compound of formula (II)

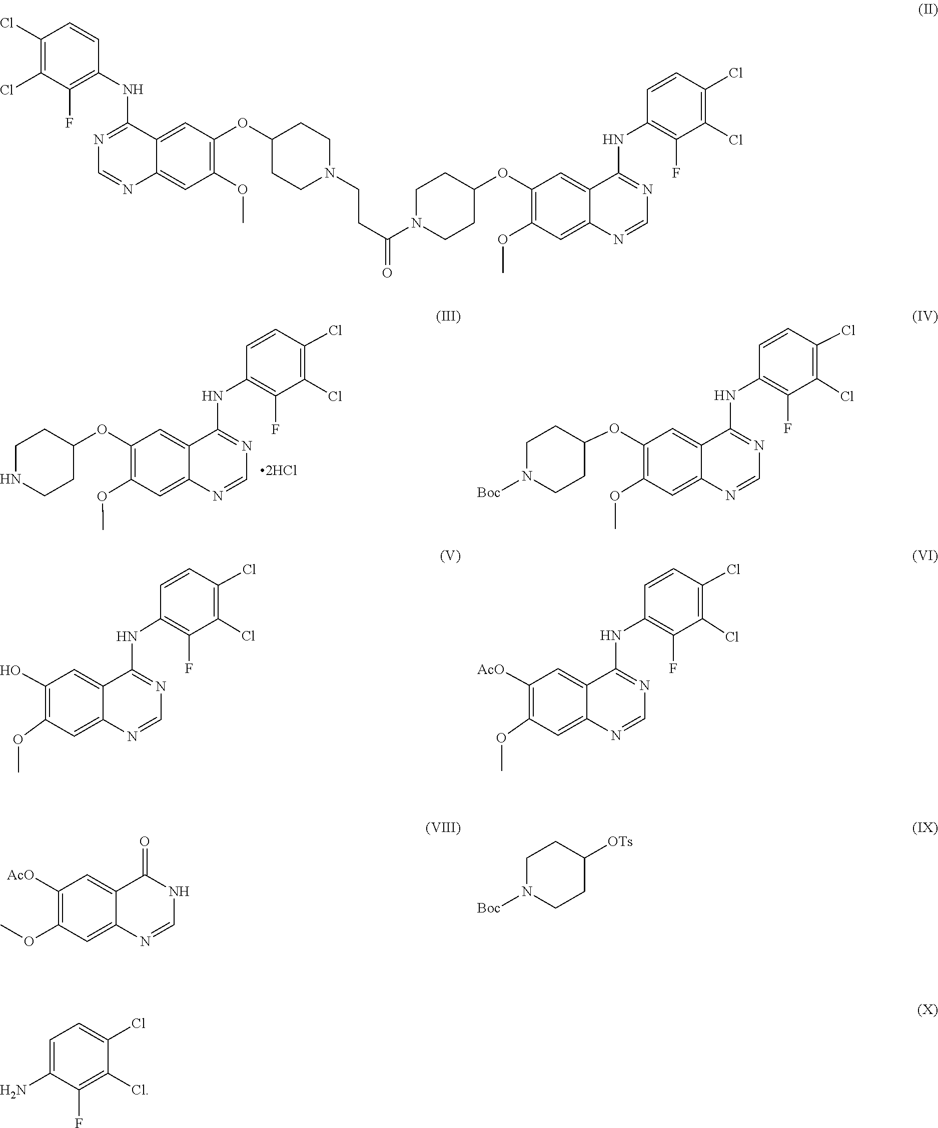

2. The method of claim 1, wherein Step (1) is conducted in a solvent selected from the group consisting of toluene, benzene and a mixture thereof.

3. The method of claim 1, wherein said organic base in Step (1) is selected from the group consisting of diisopropylamine, triethylamine, diisopropyl ethylamine, diethylamine, pyridine, 4-dimethylpyridine, morpholine and a mixture thereof.

4. The method of claim 1, wherein said halogenating agent in Step (1) is selected from the group consisting of thionyl chloride, phosphorusoxy chloride and a mixture thereof.

5. The method of claim 1, wherein said polar protic solvent in Step (2) is selected from the group consisting of methanol, ethanol, propanol and a mixture thereof.

6. The method of claim 1, wherein said inert polar aprotic solvent in Step (3) further comprises a member selected from the group consisting of N,N-dimethylformamide, N,N-dimethylacetamide, N-methylpyrrolidin-2-one, dimethyl sulfoxide and a mixture thereof.

7. The method of claim 1, wherein said base in Step (3) is an alkali metal carbonate salt selected from the group consisting of sodium hydrogen carbonate, potassium carbonate, cesium carbonate and a mixture thereof.

8. The method of claim 7, wherein said base is employed in an amount of 1 to 5 mole equivalents based on 1 mole equivalent of the compound of formula (V).

9. The method of claim 1, wherein Step (3) further comprises recrystallizing the compound of formula (IV) with acetone.

10. The method of claim 1, wherein said inert solvent in Step (4) is selected from the group consisting of methanol, ethanol, propanol, ethyl acetate, methyl acetate, acetone and a mixture thereof.

11. The method of claim 1, wherein said acid in Step (4) is employed in an amount of 3 to 10 mole equivalents based on 1 mole equivalent of the compound of formula (IV).

12. The method of claim 1, wherein the acid of Step (4) is hydrochloric acid.

13. The method of claim 1, wherein Step (5) is conducted in an organic solvent selected from the group consisting of tetrahydrofuran, ethyl acetate, acetone, 1,4-dioxane, acetonitrile, dichloromethane, carbon tetrachloride, chloroform, N,N-dimethyl formamide and dimethylsulfoxide, or a mixture of said organic solvent and water.

14. The method of claim 1, wherein said base in Step (5) is selected from the group consisting of sodium carbonate, calcium carbonate, potassium carbonate, sodium hydroxide, potassium hydroxide, cesium carbonate, diisopropylamine, triethylamine, diisopropylethylamine and diethylamine.

15. The method of claim 1, wherein said base in Step (5) is employed in an amount of 3 to 5 mole equivalents based on 1 mole equivalent of the compound of formula (III).

16. The method of claim 1, further comprising reacting the compound of Formula (II) with an acid to form a salt.

17. The method of claim 16, wherein the acid is hydrochloric acid.
